(19) United States
(12) Patent Application Publication
(10) Pub. No.: US 2009/0177154 A1
(43) Pub. Date: Jul. 9, 2009

(54) INSULIN PUMP WITH CONVENIENCE FEATURES

(76) Inventor: Michael Blomquist, Blaine, MN (US)

Correspondence Address: SCHWEGMAN, LUNDBERG & WOESSNER, P.A., P.O. BOX 2938, MINNEAPOLIS, MN, 55402, US

(21) Appl. No.: 11/970,691

(22) Filed: Jan. 8, 2008

(51) Int. Cl. A61M 37/00; G01N 33/48; A61M 31/00

(52) U.S. Cl. 604/131; 702/19; 604/66

(57) ABSTRACT

This document discusses, among other things, an apparatus comprising a pump configured to deliver insulin, a user interface including a display, and a controller communicatively coupled to the pump and the user interface. The controller includes a site display module configured to display a next suggested location of the body for the user to use in treating diabetes from a series of suggested body locations.

### BACKGROUND

People who suffer from diabetes require insulin to keep their blood glucose level as close as possible to normal levels. It is essential for people with diabetes to manage their blood glucose level to within a normal range. Complications from diabetes can include heart disease (cardiovascular disease), blindness (retinopathy), nerve damage (neuropathy), and kidney damage (nephropathy). Insulin is a hormone that reduces the level of blood glucose in the body. Normally, insulin is produced by beta cells in the pancreas. In non-diabetic people, the beta cells release insulin to satisfy two types of insulin needs. The first type is a low-level of background insulin that is released throughout the day. The second type is a quick release of a higher-level of insulin in response to eating. Insulin therapy replaces or supplements insulin produced by the pancreas.

Conventional insulin therapy typically involves one or two injections a day. The low number of injections has the disadvantage of allowing larger variations in a person's insulin levels. Some people with diabetes manage their blood glucose level with multiple daily injections (MDI). MDI may involve more than three injections a day and four or more blood glucose tests a day. MDI offers better control than conventional therapy. However, insulin injections are inconvenient and require a diabetic person to track the insulin doses, the amount of carbohydrates eaten, and their blood glucose levels among other information critical to control.

It is important for a diabetic person to be treated with the proper amount of insulin. As discussed previously, high blood sugar can lead to serious complications. Conversely, a person with low blood sugar can develop hypoglycemia. Ideally, insulin therapy mimics the way the body works. An insulin pump is one way to mimic the body's insulin production. An insulin pump can provide a background or basal infusion of insulin throughout the day and provide a quick release or bolus of insulin when carbohydrates are eaten. If a person develops high blood sugar, a correction bolus can be delivered by the pump to correct it. While insulin pumps improve convenience and flexibility for a diabetic person, they can be sophisticated devices. Some insulin pumps can be difficult to program. Proper use of an insulin pump requires a user to go through a learning curve to properly treat their diabetes using the insulin pump.

### OVERVIEW

This document discusses, among other things, devices and methods for managing insulin therapy. An apparatus example includes a pump configured to deliver insulin, a user interface including a display, and a controller communicatively coupled to the pump and the user interface. The controller includes a site display module configured to display a next suggested location of the body for the user to use in treating diabetes from a series of suggested body locations.

A method example includes providing insulin therapy to a user of a device that includes an insulin pump, and displaying, with the device, a next suggested location of the body for the user to use in treating diabetes from a series of suggested body locations.

This summary is intended to provide an overview of the subject matter of the present patent application. It is not intended to provide an exclusive or exhaustive explanation of the invention. The detailed description is included to provide further information about the subject matter of the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, examples of various embodiments discussed in the present document.

FIGS. 1A and 1B illustrate portions of a device that includes an insulin pump.

FIG. 2 is a block diagram of portions of an embodiment of a device to automatically suggest to a user a location of the body to use in treating their diabetes.

FIG. 3 shows a flow diagram of an example of a method to assist an insulin pump user in managing diabetes treatment sites.

FIG. 4 is block diagram of portions of an embodiment of a device to provide a vision test for an insulin pump user.

FIG. 5 shows an example of an Amsler Grid.

FIG. 6 shows a flow diagram of a method to assist a diabetic in managing their diabetes using an insulin pump device.

FIG. 7 is block diagram of portions of an embodiment of a device to calculate projected insulin use by the pump user.

FIG. 8 is a flow diagram of a method of managing insulin therapy using an insulin pump device.

FIG. 9 is a block diagram of portions of an embodiment of a device to record operation of the device as a log in memory.

FIG. 10 shows an example display of events related to insulin delivery.

FIG. 11 is a block diagram of portions of another embodiment of a device to record operation of the device as a log in memory.

FIG. 12 is a flow diagram of another example of a method of managing insulin therapy using an insulin pump device.

### DETAILED DESCRIPTION

Insulin Pumps can be sophisticated devices. Insulin pumps that help coach a person in the use of the device may cause the device to be more effective in treating a person's diabetes.

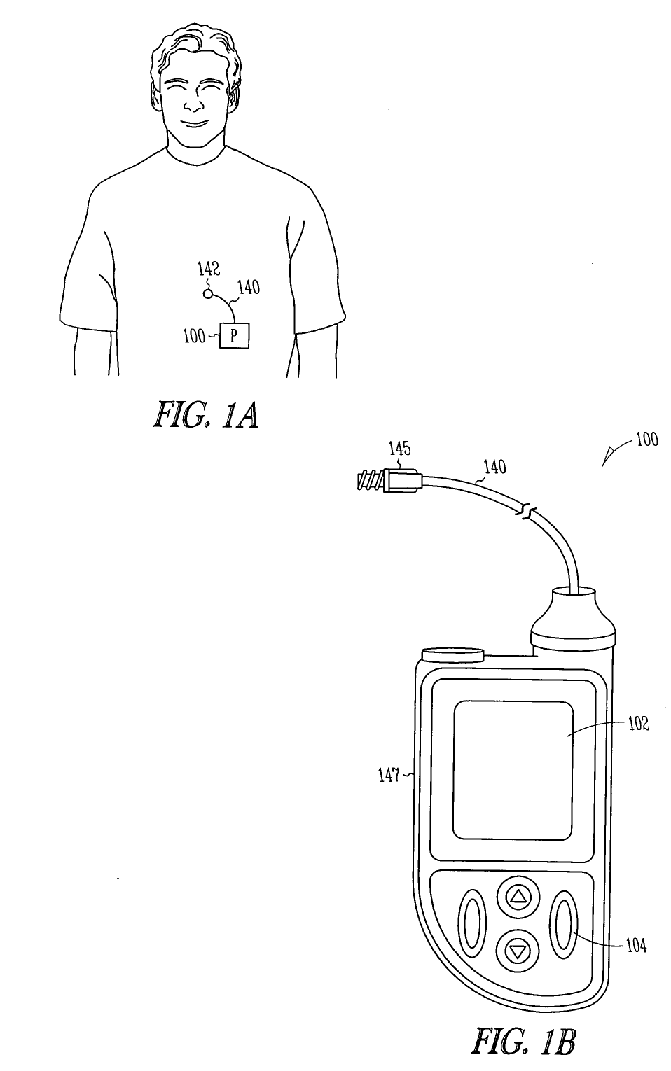

FIGS. 1A and 1B illustrate portions of a device 100 that includes an insulin pump. The device 100 includes a cassette or cartridge of insulin. The cartridge is connectable to infusion tubing 140 connectable to a patient such as by a Luer lock 145 or infusion set 142. The device 100 includes a display 102 and a user interface that may include the display 102 and include one or more keys 104 in a keypad. Because it is important for an insulin pump user to properly treat their diabetes using the pump, it is desirable for a pump to have features that make the pump more convenient to use.

### Managing Blood Glucose Test Sites and Infusion Sites

It is important that an insulin pump user rotate the site of their infusion set (i.e., the infusion site). Without proper rotation, an infusion site can become infected. It is also important that an insulin pump user rotate the site at which they test their blood glucose. If the diabetic is using a stick method to test their blood glucose, this allows the test site to heal and prevents excessive callusing or scarring.

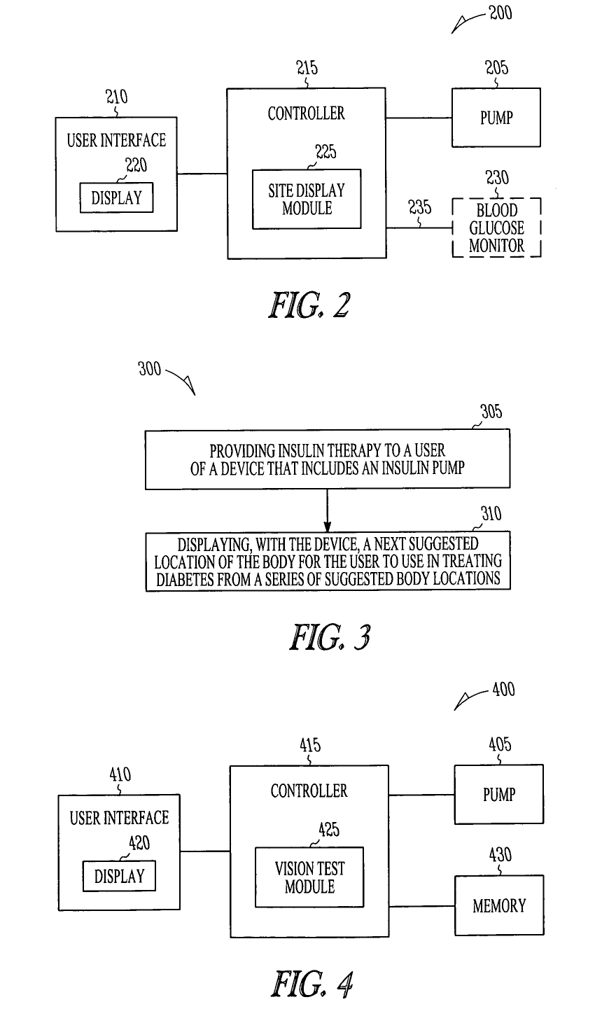

FIG. 2 is a block diagram of portions of an embodiment of a device 200 to automatically suggest a location of the body to use in treating their diabetes. The suggested location may be the next suggested infusion site for the user and/or may the next suggested blood glucose testing site. The device 200 includes a pump 205 configured to deliver insulin, a user interface 210, and a controller 215.

The pump 205 may be a positive displacement pump. Descriptions of an example of a medication pump to deliver insulin are found in Vilks et al., “Cartridge and Rod for Axially Loading a Medication Pump,” U.S. Pat. No. 7,033,338, filed Feb. 28, 2002, which is incorporated herein by reference in its entirety. The pump 205 may drive a plunger in a removable insulin cartridge to deliver the insulin.

The user interface 210 may be any interface circuit or circuits that allow a pump user to interact with the device 200. Examples include, without limitation, a keypad or a touch-screen. The user interface includes a display 220 to provide a location of the body using text and/or a picture graphic of the location.

The controller 215 can be implemented using hardware circuits, firmware, software or any combination of hardware, firmware, and software. Examples, include a microcontroller, a logical state machine, a field programmable gate array (FPGA), application specific integrated circuit (ASIC), and a processor such as a microprocessor, digital signal processor, or other type of processor. The controller 215 is configured to perform or execute a function or functions. Such functions correspond to modules, which are software, hardware, firmware or any combination thereof. Multiple functions may be performed in one or more modules. In some examples, software or firmware is provided on a computer readable medium. The computer readable medium includes instructions therein, which when processed (such as by the controller 215 for example) results in a device performing the functions described herein. Examples of a computer readable medium include a compact disc (CD), memory stick, or remote storage accessible via a communication network such as the internet or a cell phone network.

The pump 205, the user interface 210, and the controller 215 are communicatively coupled and are arranged to communicate using electrical signals that influence the operation of the devices. In some examples, the devices are coupled directly. In some examples, the devices communicate electrical signals through intermediate digital or analog circuits.

The controller 215 includes a site display module 225 configured to display via the user interface a next suggested location, or site, of the body for the user to use in treating diabetes from a series of suggested body locations. In some examples, the site display module 225 displays a graphic of the suggested body location. In some examples, the site display module 225 displays a code indicating the suggested body location (e.g., “RTL” for right thumb of the left hand).

In some embodiments, the site display module 225 displays a next suggested infusion site for the user from a series of suggested infusion sites. The series of infusion sites may be stored in a memory integral to, or communicatively coupled to, the controller 215. In some embodiments, the site display module 225 selects the next infusion site for display to the user by rotating through a series of body infusion site locations. Typically, an insulin pump user should change the infusion site every three days. The suggested infusion sites may include, among others, locations of one or more of an abdomen, shoulder, thigh, or buttock. In some embodiments, the user is able to remove and/or add sites to the series of body sites to be displayed. Thus, the series of body locations is a subset of possible body locations selectable by a user. This allows the user to input a preference for the series of body infusion sites. The site display module 225 rotates the next suggested site only through locations previously indicated by the user as desirable for treating diabetes. In some embodiments, the site display module 225 displays the next suggested infusion site when an insulin cartridge is loaded into the device 200 and connected to the pump 205.

According to some embodiments, the site display module 225 displays a next suggested location of a next blood glucose test site for the user from a series of blood glucose test sites. In some embodiments, the site display module 225 selects the next suggested blood glucose test site for display by rotating through a series of body test site locations for display. The suggested blood glucose test sites may include locations on the fingers or thumb of the hands and/or other test sites such as on the forearms. In some examples, the device 200 includes a blood glucose monitor 230 communicatively coupled to an input 235 of the controller 215. The site display module 225 is configured to display the next suggested location of a next blood glucose test site when a blood glucose test strip is inserted into the device 200.

In some embodiments, the controller 215 is configured to execute a plurality of features selectable via a menu displayed on the user interface 210. The site display module 225 displays the next suggested location of a next blood glucose test site when a menu choice, displayed on the insulin pump device, is selected that requires a blood glucose measurement. For example, the controller 215 may be configured to calculate a carbohydrate ratio, to run a correction factor test, or to run a basal rate test.

Descriptions of devices and methods that perform a carbohydrate ratio test are found in Blomquist, “Carbohydrate Ratio Testing Using Frequent Blood Glucose Input,” U.S. patent application Ser. No. 11/679,712, filed Feb. 27, 2007, which is incorporated herein by reference in its entirety. Descriptions of devices and methods that perform a correction factor test are found in Blomquist et al., “Correction Factor Testing Using Frequent Blood Glucose Input,” U.S. patent application Ser. No. 11/626,653, filed Jan. 24, 2007, which is incorporated herein by reference in its entirety. Descriptions of devices and methods that perform a basal rate test are found in Blomquist et al., “Basal Rate Testing Using Frequent Blood Glucose Input,” U.S. patent application Ser. No. 11/685,617, filed Mar. 13, 2007, which is incorporated herein by reference in its entirety. When the calculation or test needs a blood glucose measurement, the site display module 225 displays a suggested location for the blood glucose test site.

FIG. 3 shows a flow diagram 300 of a method to assist an insulin pump user in managing diabetes treatment sites. At block 305, insulin therapy is provided to a user of a device that includes an insulin pump. At block 310, the device displays a next suggested location of the body for the user to use in treating diabetes from a series of suggested body locations. The suggested location may be for an infusion site, or the suggested location may be for a blood glucose test site.

### Vision Test

As stated previously, complications from diabetes can include retinopathy or other retinal disorders. FIG. 4 is block diagram of portions of an embodiment of a device 400 to provide a vision test for an insulin pump user. The device 400 includes a pump 405 configured to deliver insulin, a user interface 410, and a controller 415. The user interface includes a display 420. The controller 415 includes a vision test module 425. The vision test module 425 executes a test to monitor a central visual field of a user. Such a test is useful to detect or track retinal disorders of the user.

In some embodiments, the vision test module 425 displays an Amsler Grid on the display 420. FIG. 5 shows an example of an Amsler Grid 500. In some embodiments, the vision test module 425 displays instructions on the display 420 for using the Amsler Grid 500. For example, the vision test module 425 may display instructions such as directing the pump user to cover one eye while looking at the display 420 and asking whether the Amsler Grid 500 appears distorted, whether any lines look wavy or crooked, whether any lines are missing, or whether all the boxes in the Amsler Grid 500 look the same. The vision test module 425 may display instructions to contact an eye doctor if the user perceives distortion in the Amsler Grid 500.

In some embodiments, the device 400 includes a memory 430 integral to, or communicatively coupled to, the controller 415. The pump user enters grid coordinates of any perceived distortion into the device 400 via the user interface 410. The controller 415 receives a coordinate of the Amsler Grid 500 via the user interface, stores the grid coordinate entered via the user interface 410 into the memory 430 as indicating a location of visual distortion. The controller 415 may later display any grid coordinates, stored over a period of time, which indicate visual distortion.

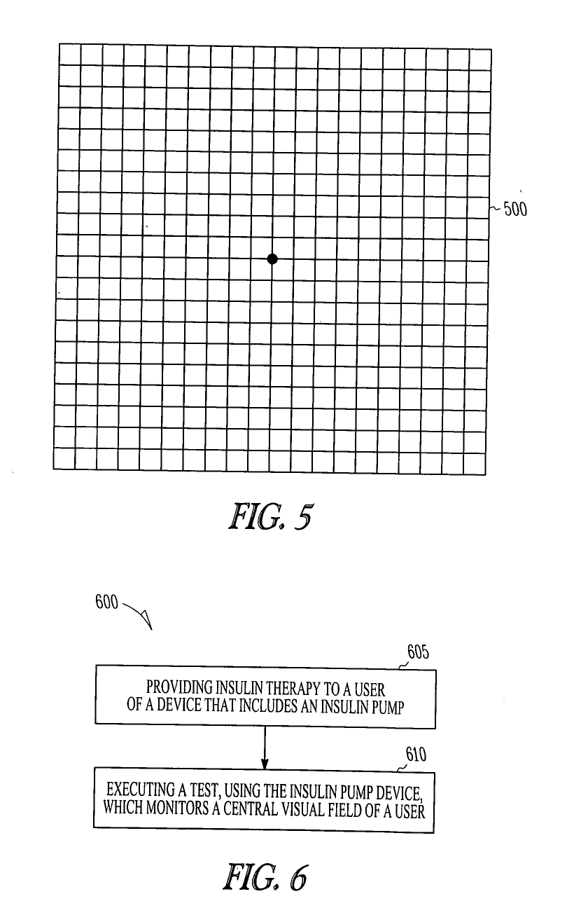

FIG. 6 shows a flow diagram of a method 600 to assist a diabetic in managing their diabetes using an insulin pump device. At block 605, insulin therapy is provided to a user of a device that includes an insulin pump. At block 610, a test is executed by the insulin pump device which monitors a central visual field of a user.

Returning to FIG. 4, in some embodiments, the controller 415 generates a report that includes any grid coordinates that indicate visual distortion. The report may be displayed via the display 420. In certain embodiments, the device 400 may include a communication port communicatively coupled to the controller 415. The communication port may be a wired port (e.g., universal serial bus port) or a wireless port (e.g., a radio frequency or an infrared port). The report is communicated from the insulin pump device to a second separate device via the communication port. The second separate device may include a printer to print the report, or may be a computer to display the report. By tracking grid coordinates that indicate visual distortion, the user or diabetes professional is able to track any distortion over time to monitor the progress of retinopathy or other retinal disorder.

According to some embodiments, the vision test module 425 periodically displays a prompt to the user to execute the test to monitor the user's central visual field. In some embodiments, the vision test module 425 displays the prompt in a time relationship to a bolus delivered using the insulin pump device. In some embodiments, the vision test module 425 displays the prompt in a time relationship to receiving a blood glucose measurement into the device 400 via the user interface 410. In some embodiments, the vision test module 425 displays the prompt in a time relationship to when an insulin cartridge is changed on the insulin pump device 400.

In some embodiments, the vision test module 425 displays the prompt according to a test schedule stored in the memory 430. For example, the test schedule may prompt the user to look at an Amsler Grid, or other test for the user's central visual field, once per month. The test schedule may be entered into the device 400 via the user interface 410, or the device 400 may include a communication port and the test schedule is downloaded into the device 400 from a second separate device such as a laptop computer for example.

### Calculating Insulin Usage

An insulin pump device typically includes a cartridge to hold insulin. An insulin pump empties insulin from the cartridge to deliver insulin therapy to the pump user. The pump user changes or refills the cartridge with insulin when it is emptied. There may be times when it is inconvenient for the user to refill the cartridge. For example, the user may be traveling or may have to attend an extended business meeting. The user may have to estimate whether the insulin cartridge will empty during such a time and refill the cartridge accordingly. The user has to pay attention to the level of the insulin cartridge to avoid an inconvenient filling time, or else the user has to estimate the amount of insulin that will needed to get them past the inconvenient filling time and fill the cartridge accordingly. It would be desirable to have the insulin pump device to track the amount of insulin being used, and assist the user estimating the amount of insulin needed.

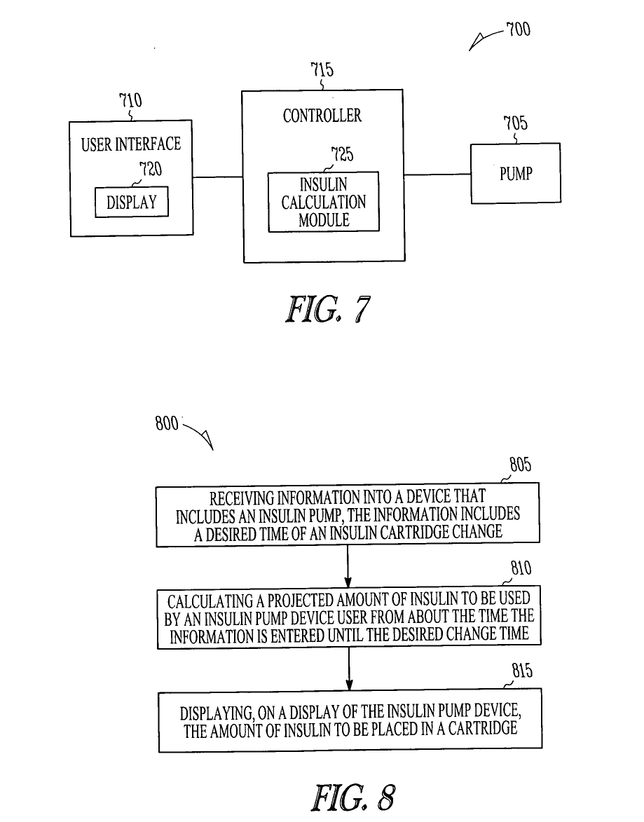

FIG. 7 is block diagram of portions of an embodiment of a device 700 to calculate projected insulin use by the pump user. The device 700 includes a pump 705 configured to deliver insulin, a user interface 710, and a controller 715. The insulin pump 705 removes insulin from an insulin cartridge to deliver the insulin. The user interface includes a display 720. The controller 715 includes an insulin calculation module 725 to calculate insulin user by the pump user. The insulin calculation module 725 receives a desired time for changing an insulin cartridge via the user interface 710. The insulin calculation module 725 calculates an amount of insulin using the desired change time, and displays the amount of insulin to be placed in the cartridge on the display 720.

To calculate the amount of insulin, the insulin calculation module 725 monitors historical use patterns of the user and monitors the amount of insulin used after a cartridge change. For example, if the user enters information via the user interface 710 that they want to fill the insulin cartridge at that time with enough insulin to last over a two-day business trip, the insulin calculation module adds up the insulin historically used for two days or historically used for those two days of the week of the business trip and displays the amount of insulin to put in the insulin cartridge. In adding up the insulin historically used, the insulin pump may add together the basal insulin delivered during the indicated time period, the amount of insulin in any meal or carbohydrate boluses typically delivered, and the amount of insulin in correction boluses typically delivered during the period. The historical insulin use of the pump user may be stored in a memory integral to, or communicatively coupled to, the controller 715.

The time (or about the time) of the cartridge change may be entered by the user via the user interface or the time of the cartridge change may be detected using a change detection circuit in the device 700. The insulin calculation module 725 monitors the insulin use after the cartridge change to determine the amount of insulin remaining in the cartridge.

According to some embodiments, the insulin calculation module 725 calculates a time that an insulin cartridge will empty. Based on the indicated time that the insulin cartridge was filled, the amount of insulin in the cartridge, and the historical insulin use of the pump user, the insulin calculation module 725 determines the time that the cartridge will empty. The insulin calculation module 725 displays the projected time on the user interface 710 of the device 700. In some examples, the insulin calculation module 725 calculates the projected time in response to a prompt received via the user interface 710. In some examples, the insulin calculation module 725 calculates the expected time the cartridge will empty when the device 700 receives an indication that the cartridge is changed or filled, and displays the time when a prompt is received through the user interface 710. In some examples, the insulin calculation module 725 keeps updating the expected time the cartridge will empty and displays the time when the prompt to do so is received.

In some examples, the pump user enters one or more times a cartridge change is preferred or one or more times a cartridge change is not preferred. When a prompt is received via the user interface 710 to initiate a delivery of a bolus of insulin, the insulin calculation module 725 calculates the amount of insulin in the bolus and compares the amount to an amount of insulin remaining in the cartridge. The insulin calculation module 725 displays a warning if the delivery of the insulin bolus would require a change of the insulin cartridge outside of the desired time or times entered by the pump user. For example, the user may prefer not to change the cartridge between midnight and 6:00 AM and may forego a bedtime snack to allow the cartridge to last through the night. In some examples, the user may initiate the insulin bolus despite the warning.

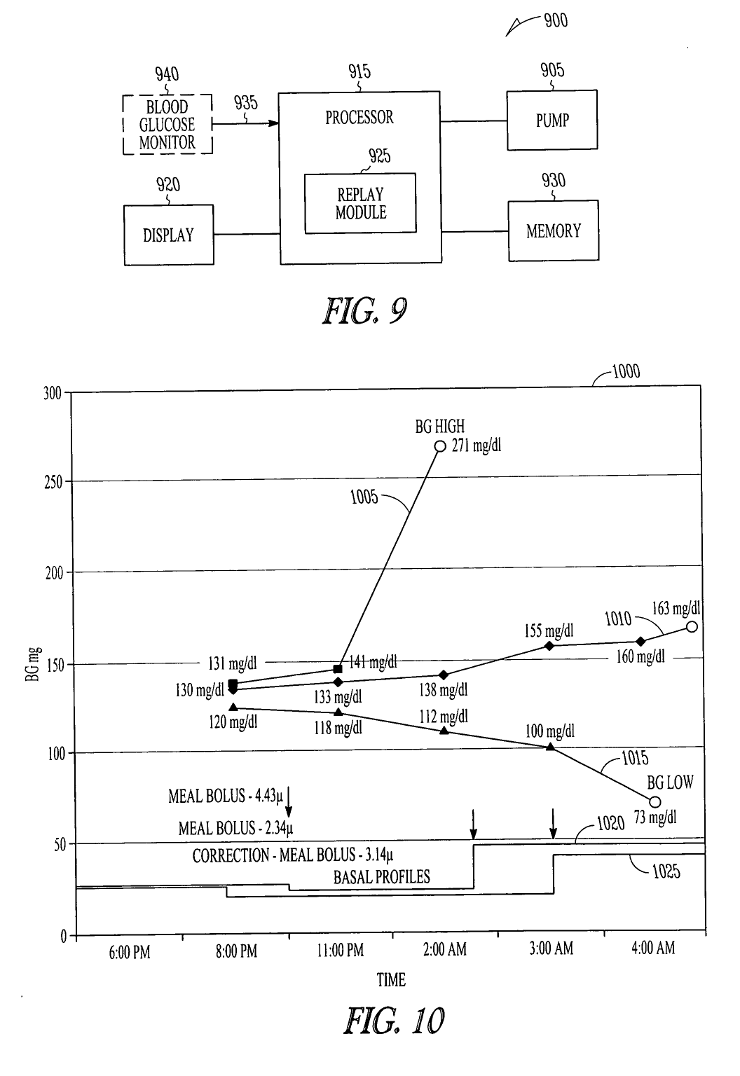

FIG. 8 is a flow diagram of a method 800 of managing insulin therapy using an insulin pump device. At block 805, information is received into an insulin pump device. The information includes a desired time of an insulin cartridge change. At block 810, calculating a projected amount of insulin to be used by an insulin pump device user is calculated from about the time the information is entered to the desired change time. At block 815, the amount of insulin to be placed in a cartridge is displayed.

### Device History Log

A pump user may go through several iterations of trial and error in finding appropriate insulin pump settings. For example, basal rate refers to a background infusion of insulin provided throughout the day by an insulin pump device. Sometimes half a user's total daily dose (TDD) of insulin is delivered in this manner. Setting basal rate patterns to maintain blood glucose within a healthy range is often an iterative process. Tracking efficacy of the insulin therapy provided by the pump would aid the user or diabetes professional in properly programming the insulin pump device for the user.

FIG. 9 is a block diagram of portions of an embodiment of a device 900 to record operation of the device 900 as a log in memory. The device 900 includes a pump 905 configured to deliver insulin, an input 935 to receive blood glucose data that indicates a blood glucose level of the user, a display 920, and a processor 915. The device 900 also includes a memory 930 integral to, or communicatively coupled to, the processor 915. The memory 930 stores indications of a plurality of events related to insulin therapy as a memory log.

The processor 915 includes a replay module 925 that replays the events on the display 920 as they occurred in time by displaying a moving segment of the received blood glucose data together with the stored indications.

FIG. 10 shows an example display 1000 of events related to insulin delivery. The display 1000 shows three different hypothetical examples of displaying overnight blood glucose levels. The first example 1005 shows a high blood glucose level, the second example 1015 shows a low blood glucose level, and the third example, 1010 shows blood glucose maintained within a healthy range. In some embodiments, the replay module 925 displays a graph of a segment of blood glucose data that includes an amount of insulin delivered. In some embodiments, the processor 915 of FIG. 9 stores an indication of a blood glucose level of the user exceeding a threshold blood glucose value or dropping below a threshold value, and displays the indication.

The example display 1000 also shows basal rate profiles 1020 and 1025, or basal rate patterns, delivered during the overnight period. The replay module 925 displays a moving segment (e.g., from left to right) of the received blood glucose data together with the stored indications of the plurality of events related to insulin delivery. Replaying blood glucose data makes it easier for a user to follow the effect different events had on the blood glucose level.

In some embodiments, the processor 915 stores in the memory 930 an indication of an event as an event-identifying marker in a time relationship to the blood glucose data. For example, the display 1000 in FIG. 10 shows example markers for two meal boluses of insulin and a correction bolus of insulin plus a meal bolus of insulin. The event-identifying markers are shown in a time relationship to the blood glucose data. In some embodiments, the processor 915 stores an indication of a delivery of a change in basal rate as the event-identifying marker. In FIG. 10 the display shows two basal profiles and indicates the change in a basal rate.

According to some embodiments, the device 900 of FIG. 9 includes a blood glucose monitor 940 communicatively coupled to the input 935. In certain examples, the blood glucose monitor is a continuous blood glucose monitor that automatically collects the blood glucose data. A continuous blood glucose monitor may include a blood glucose sensor circuit to produce an electrical blood glucose signal representative of a blood glucose level of the patient. A description of a blood glucose sensor circuit can be found in Steil et al., “Closed Loop System for Controlling Insulin Infusion,” U.S. Pat. No. 6,558,351, filed Jun. 1, 2000, which is incorporated herein by reference in its entirety.

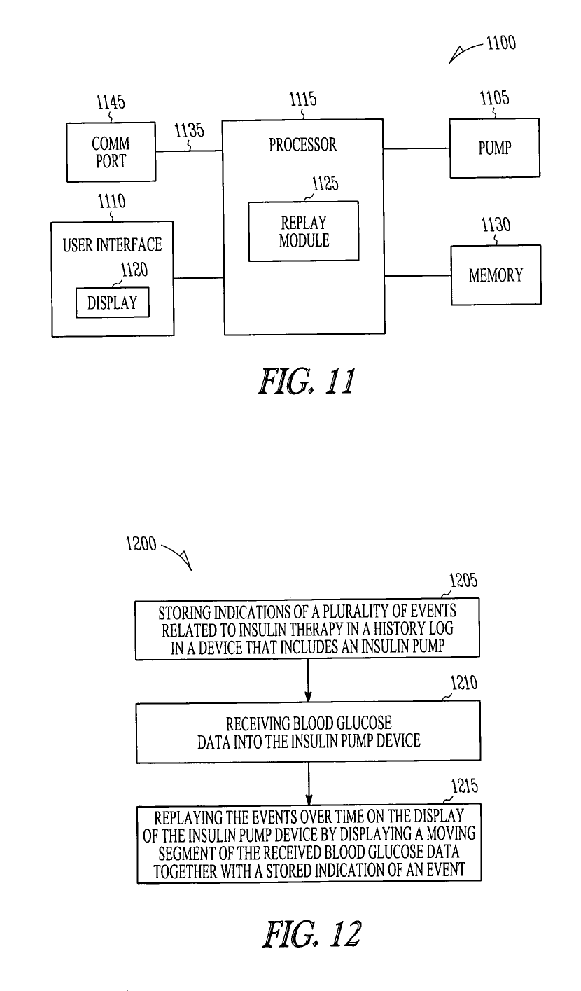

FIG. 11 is a block diagram of portions of another embodiment of a device 1100 to record operation of the device 1100 as a log in memory. The device 1100 includes a pump 1105 configured to deliver insulin, an input 1135 to receive blood glucose data, a user interface 1110 that includes a display 1120, and a processor 1115. The device 1100 also includes a memory 1130 to store indications of events as a memory log and a replay module 1125.

The user interface 1110 allows a pump user to interact with the device 1100. In some embodiments, the processor 1115 prompts the user, via the display 1120, to begin a blood glucose measurement using a second separate device. In some embodiments, the user interface 1110 and the input 1135 are configured to receive the blood glucose data entered manually by the user. In some embodiments, the processor 1115 is configured to periodically prompt a user, via the display 1120, to manually enter the blood glucose measurement via the user interface 1110. The user then enters the blood glucose measurement.

In some embodiments, the device 1100 includes a communication port 1145 communicatively coupled to the input 1135. The processor 1115 receives the blood glucose data from a second separate device via the communication port 1145. The communication port 1145 may be a wired port or a wireless port.

FIG. 12 is a flow diagram of another example of a method 1200 of managing insulin therapy using an insulin pump device. At block 1205, indications of a plurality of events related to insulin therapy are stored in a history log in a device that includes an insulin pump. At block 1210, blood glucose data is received into the insulin pump device. The blood glucose data indicates a blood glucose level of a user of the insulin pump device. At block 1215, the events are replayed over time on a display of the insulin pump device by displaying a moving segment of the received blood glucose data together with a stored indication of an event. In some embodiments, replaying the events as they occur in a history log includes animating the lag between a delivery of insulin and the effect on the user's blood glucose. The delivery may include a meal bolus, a correction bolus, and/or a change to basal insulin delivery pattern.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. In this document, the terms “a” or “an” are used, as is common in patent documents, to include one or more than one. In this document, the term “or” is used to refer to a nonexclusive or, such that “A or B” includes “A but not B,” “B but not A,” and “A and B,” unless otherwise indicated.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own.

What is claimed is:

1. An apparatus comprising:
a pump configured to deliver insulin;
a user interface including a display; and
a controller communicatively coupled to the pump and the user interface, wherein the controller includes a site display module configured to display a next suggested location of the body for the user to use in treating diabetes from a series of suggested body locations.

2. The apparatus of claim 1, wherein the site display module is configured to display a next suggested infusion site for the user from a series of suggested infusion sites.

3. The apparatus of claim 2, wherein the site display module is configured to display the next suggested infusion site for the user by rotating through a series of body infusion site locations for display.

4. The apparatus of claim 3, wherein the series of body locations is a subset of possible body locations selectable by a user.

5. The apparatus of claim 2, wherein the site display module is configured to display the next suggested infusion site when an insulin cartridge is connected to the pump.

6. The apparatus of claim 1, wherein the site display module is configured to display a next suggested location of a next blood glucose test site for the user from a series of blood glucose test sites.

7. The apparatus of claim 6, wherein the site display module is configured to select the next suggested blood glucose test site for display by rotating through a series of body test site locations for display.

8. The apparatus of claim 6, including a blood glucose monitor, communicatively coupled to the controller, and wherein the site display module is configured to display the next suggested location of a next blood glucose test site when a blood glucose test strip is inserted into the insulin pump device.

9. The apparatus of claim 6, wherein the controller is configured to execute a plurality of features selectable via a menu displayed on the user interface, and wherein the site display module is configured to display the next suggested location of a next blood glucose test site when a menu choice is selected that requires a blood glucose measurement.

10. The apparatus of claim 1, wherein the site display module is configured to display a graphic of the suggested body location.

11. The apparatus of claim 1, wherein the site display module is configured to display a code indicating the suggested body location.

12. The apparatus of claim 1, wherein the site display module is configured to rotate the next suggested site only through locations previously indicated by the user as desirable for treating diabetes.

13. A method comprising:
providing insulin therapy to a user of a device that includes an insulin pump; and
displaying, with the device, a next suggested location of the body for the user to use in treating diabetes from a series of suggested body locations.

14. The method of claim 13, wherein displaying a next suggested location of the body includes displaying a suggested location of a next infusion site for the user.

15. The method of claim 14, wherein displaying the suggested location of the next infusion site includes displaying the suggested location of the next infusion site when a cartridge is loaded into the insulin pump device.

16. The method of claim 14, wherein displaying the suggested location of the next infusion site includes selecting the suggested site for display by rotating through a series of body infusion site locations.

17. The method of claim 16, wherein rotating through the series of body infusion site locations includes rotating through a subset of possible body locations selected by the user.

18. The method of claim 13, wherein displaying a next suggested location of the body includes displaying a suggested location of a next blood glucose test site for the user.

19. The method of claim 18, wherein displaying the suggested location of the next blood glucose test site includes displaying the location of the next blood glucose test site when a blood glucose test strip is inserted into the insulin pump device.

20. The method of claim 18, wherein displaying the suggested location of the next blood glucose test site includes displaying the suggested location of the next blood glucose test site when a menu choice, displayed on the insulin pump device, is selected that requires a blood glucose measurement.

21. The method of claim 18, wherein displaying the suggested location of the next blood glucose test site includes selecting the suggested site for display by rotating through a series of body test site locations.

22. The method of claim 13, wherein displaying a next suggested location of the body includes displaying a graphic of the body location.

23. The method of claim 13, wherein displaying a next suggested location of the body includes displaying a code indicating the body location.

24. The method of claim 13, wherein displaying a next suggested location of the body includes rotating the suggested site through only those locations indicated as desirable by the user for treating diabetes.

25. An apparatus comprising:
a pump configured to deliver insulin;
a user interface including a display; and
a controller communicatively coupled to the pump and the user interface, wherein the controller includes a vision test module configured to execute a test to monitor a central visual field of a user.

26. The apparatus of claim 25, wherein the vision test module is configured to display an Amsler Grid on the display.

27. The apparatus of claim 26, wherein the vision test module is configured to display instructions for using the Amsler Grid.

28. The apparatus of claim 26, including a memory communicatively coupled to the controller, wherein the controller is configured to:
receive a coordinate of the Amsler Grid via the user interface;
store the grid coordinate in the memory as indicating a location of visual distortion; and
display any grid coordinates, stored over a period of time, which indicate visual distortion.

29. The apparatus of claim 25, wherein the vision test module is configured to periodically display a prompt to the user to execute the test to monitor a central visual field of the user.

30. The apparatus of claim 29, wherein the vision test module is configured to display the prompt in a time relationship to a bolus delivered using the insulin pump device.

31. The apparatus of claim 29, wherein the vision test module is configured to display the prompt in a time relationship to receiving a blood glucose measurement into the insulin pump device.

32. The apparatus of claim 29, wherein the vision test module is configured to display the prompt in a time relationship to when an insulin cartridge is changed on the insulin pump device.

33. The apparatus of claim 29, including a memory communicatively coupled to the controller, wherein the vision test module is configured to display the prompt according to a test schedule stored in the memory.

34. A method comprising:
providing insulin therapy to a user via a device that includes an insulin pump; and
executing, with the insulin pump device, a test to monitor a central visual field of a user.

35. The method of claim 34, wherein displaying the test to monitor the central visual field includes displaying an Amsler Grid on the insulin pump device.

36. The method of claim 35, including displaying, on the insulin pump device, instructions for using the Amsler Grid.

37. The method of claim 35, including:
receiving a coordinate of the Amsler Grid into the insulin pump device;
storing the grid coordinate in the device as indicating a location of visual distortion; and
generating a report using the insulin pump device, wherein the report includes any grid coordinates that indicate visual distortion.

38. The method of claim 37, wherein generating a report using the insulin pump device includes displaying the report using the insulin pump device.

39. The method of claim 37, wherein generating a report includes communicating the grid coordinates from the insulin pump device to a second separate device.

40. The method of claim 34, including periodically displaying a prompt to the user to execute the test to monitor the central visual field.

41. The method of claim 40, wherein periodically displaying a prompt includes periodically displaying a prompt to the user to execute the test in a time relationship to a bolus delivered by the insulin pump device.

42. The method of claim 40, wherein periodically displaying a prompt includes periodically displaying a prompt to the user to execute the test in a time relationship to receiving a blood glucose measurement into the insulin pump device.

43. The method of claim 40, wherein periodically displaying a prompt includes periodically displaying a prompt to the user to execute the test in a time relationship to changing an insulin cartridge on the insulin pump device.

44. The method of claim 40, wherein periodically displaying a prompt includes periodically displaying a prompt to the user to execute the test according to a test schedule stored in the insulin pump device.

45. An apparatus comprising:
a pump configured to deliver insulin;
a user interface including a display; and
a controller communicatively coupled to the pump and the user interface, wherein the controller includes an insulin calculation module configured to:
receive a desired time for changing an insulin cartridge via the user interface;
calculate an amount of insulin to be used by an insulin pump device user by the desired change time; and
display the amount of insulin to be placed in the cartridge on the display.

46. The apparatus of claim 45, wherein the insulin calculation module is configured to:
calculate a projected time the insulin cartridge will empty; and
display the projected time on the user interface of the device.

47. The apparatus of claim 46, wherein the insulin calculation module is configured to calculate the projected time in response to a prompt received via the user interface.

48. The apparatus of claim 45, wherein the insulin calculation module is configured to:
receive a prompt to initiate a delivery of a bolus of insulin;
calculate an amount of insulin in the bolus and compare the amount to an amount of insulin remaining in the cartridge; and
display a warning if the delivery of the insulin bolus would require a change of the insulin cartridge outside of the desired time.

49. A method including:
receiving information into a device that includes an insulin pump, the information including a desired time of an insulin cartridge change;
calculating a projected amount of insulin to be used by an insulin pump device user from about the time the information is entered until the desired change time; and
displaying, on a display of the insulin pump device, the amount of insulin to be placed in a cartridge.

50. The method of claim 49 including:
calculating a projected time the insulin cartridge will empty; and
displaying the time on the user interface of the device.

51. The method of claim 50, wherein calculating a projected time the insulin cartridge will empty includes calculating the projected time in response to a prompt received at a user interface of the insulin pump device.

52. The method of claim 49 including:
receiving a prompt to initiate a delivery of a bolus of insulin;
calculating an amount of insulin in the bolus; and
displaying a warning if the delivery of the insulin bolus would require a change of the insulin cartridge outside of the desired time.
